(19) United States
(12) Patent Application Publication
(10) Pub. No.: US 2024/0347162 A1
(43) Pub. Date: Oct. 17, 2024

(54) Body Fluid Input-Output Monitoring Systems and Methods

(71) Applicant: C. R. Bard, Inc., Franklin Lakes, NJ (US)

(72) Inventors: Thomas Michael Meese, Louisville, CO (US); Hersh Ramesh Patel, Atlanta, GA (US); Dennis Joseph, Milton, GA (US); Varad Chavan, Kolhapur (IN)

(21) Appl. No.: 18/682,075

(22) PCT Filed: Aug. 8, 2022
(86) PCT No.: PCT/US22/39746

§ 371 Date: Feb. 7, 2024

Related U.S. Application Data
(60) Provisional application No. 63/233,666, filed on Aug. 16, 2021.

(51) Int. Cl. G16H 20/17; A61M 5/14; A61M 5/172; G16H 10/60

(52) U.S. Cl. CPC G16H 20/17; A61M 5/1407; A61M 5/172; G16H 10/60; A61M 2005/1726; A61M 2202/0007; A61M 2202/0014; A61M 2202/0496; A61M 2205/12; A61M 2205/18; A61M 2205/3327; A61M 2205/3379; A61M 2205/3561; A61M 2205/502; A61M 2209/084

(57) ABSTRACT

Disclosed herein is a system for monitoring the fluid balance of a patient. The system includes a fluid infusion system to deliver an infusion fluid to the patient, and a urine output (CO) system to collect and measure a UO expelled from the patient. The UO system is coupled with the fluid infusion system. A fluid input/output (I/O) logic determines fluid I/O data from fluid infusion data and CO data, rendering the I/O data on a display, and transferring I/O data to an electronic medical record. A method of monitoring a fluid balance of a patient can include: (i) calculating a fluid balance from the I/O data, (ii) comparing the fluid balance with a fluid balance limit stored in a non-transitory computer-readable storage medium, and (iii) generating an alert when the fluid balance exceeds the fluid balance limit. The method can also include generating a revised infusion order from the I/O data.

### PRIORITY

This application claims the benefit of priority to U.S. Provisional Application No. 63/233,666, filed Aug. 16, 2021, which is incorporated by reference in its entirety into this application.

### BACKGROUND

It has been shown that maintenance of an adequate fluid balance is vital to health. Inadequate fluid intake or excessive fluid loss can lead to dehydration, which in turn can affect cardiac and renal function and electrolyte management. Inadequate urine production can lead to fluid overload, renal failure and electrolyte toxicity. Attention to fluid intake and output is an important element of nursing practice. Poor fluid balance management has been identified as a contributing factor to the poor outcome of some acutely unwell hospital patients.

When the amount of fluid lost from the body is equal to the amount of fluid taken in, the body is in fluid balance. Fluid in a body is found within the body cells (intracellular), surrounding the cells (interstitial) and within the blood vessels (intravascular). It is the bodies' principal chemical component, comprising, on average, 60 percent of body weight.

Manually charting fluid input and output has been practiced in the healthcare systems for many years. The chart documents the patients' water input and output over a defined period, typically a 24-hour period. Fluid input/output charting is an important guide to clinical decisions including medication administration and prescription as well as surgical interventions. Inaccurate or inadequate charting of fluid balance can be counterproductive and, in some instances, put the patient at significant risk.

Medical staff, nurses and dieticians rely on accurate fluid balance totals in order to plan appropriate care and reduce the risk of complications that may be associated with dehydration, malnutrition and electrolyte imbalances. Studies have shown that manually charting fluid input and output is often unreliable and in accurate. Hence, there is a need to automate the fluid balance monitoring of patients.

### SUMMARY OF THE INVENTION

Briefly summarized, disclosed herein is a system for monitoring the fluid balance of a patient. The system includes a fluid infusion system configured to deliver an infusion fluid to the patient, and a urine output (UO) system configured to collect and measure a UO expelled from the patient, where the UO system is communicatively coupled with the fluid infusion system. The system further includes a non-transitory computer-readable storage medium (CRM) including fluid input/output (I/O) logic, that when executed by one or more processors of the system, performs operations that include (i) receiving fluid infusion data from the fluid infusion system, (ii) receiving UO data from the UO system, (iii) determining I/O data from the fluid infusion data and the UO data, and (iv) rendering I/O data on a display of the system.

The I/O logic operations may further include transmitting I/O data across a network to an external entity, and the external entity may include an electronic medical record.

The fluid infusion system may be coupled with the UO system via a wired connection, and the fluid infusion system and the UO system may be attached together. In some embodiments, the fluid infusion system and the UO system may be anchored to a common support structure.

In some embodiments, the UO system includes the display and/or the I/O logic. The system may further include a housing, where the fluid infusion system and the UO system are disposed within the housing.

The I/O logic operations may further include (i) calculating a fluid balance from the I/O data, (ii) comparing the fluid balance with a fluid balance limit stored in the CRM, and (iii) generating an alert when the fluid balance exceeds the fluid balance limit. The I/O logic operations may further include transmitting the alert to the external entity.

In some embodiments, the infusion data includes an infusion order, the operations include generating a revised infusion order from the I/O data, and the alert includes the revised infusion order.

The revised infusion order may include at least one of an increased or decreased a rate of infusion. The revised infusion order may also include an altered medical dose of the infusion fluid to chemically cause a change in the fluid balance. The revised infusion order may also include a different drug to be administered.

Also disclosed herein is a method for monitoring a fluid balance of a patient. The method includes (i) receiving fluid infusion data from a fluid infusion system, the fluid infusion system delivering infusion fluid to the patient, (ii) receiving urine output (UO) data from a UO system, the UO system collecting and measuring UO from the patient, (iii) determining fluid input/output (I/O) data from the fluid infusion data and the UO data, and (iv) rendering the I/O data on a display. The UO system is coupled with the fluid infusion system to define an I/O system, and the display is coupled with the IO system.

The method may further include transmitting the I/O data across a network to an external entity, and the external entity may include an electronic medical record.

The method may further include (i) calculating a fluid balance from the I/O data, (ii) comparing the fluid balance with a fluid balance limit stored in a non-transitory computer-readable storage medium of the I/O system, and (iii) generating an alert when the fluid balance exceeds the fluid balance limit. The method may further include transmitting the alert to the external entity.

In some embodiments, the method includes generating a revised infusion order from the I/O data, and providing notification to a user of the revised infusion order. The revised infusion order may include at least one of an increased or decreased a rate of infusion. In further embodiments, the revised infusion order may include an altered medical dose of the infusion fluid to chemically cause a change in the fluid balance and/or a different drug to be administered.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a first embodiment of a body fluid input/output (I/O) monitoring system, in accordance with some embodiments;

FIG. 1B illustrates an exemplary screen shot as may be rendered on the display of the system of FIG. 1A, in accordance with some embodiments;

FIG. 1C illustrates an exemplary process of monitoring the fluid balance of a patient, in accordance with some embodiments;

FIG. 2 illustrates a second embodiment of the body fluid (I/O) monitoring system, in accordance with some embodiments; and

FIG. 3 illustrates a third embodiment of the body fluid (I/O) monitoring system, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Unless defined otherwise, all technical and scientific terms used herein have the same meaning as commonly understood by those of ordinary skill in the art.

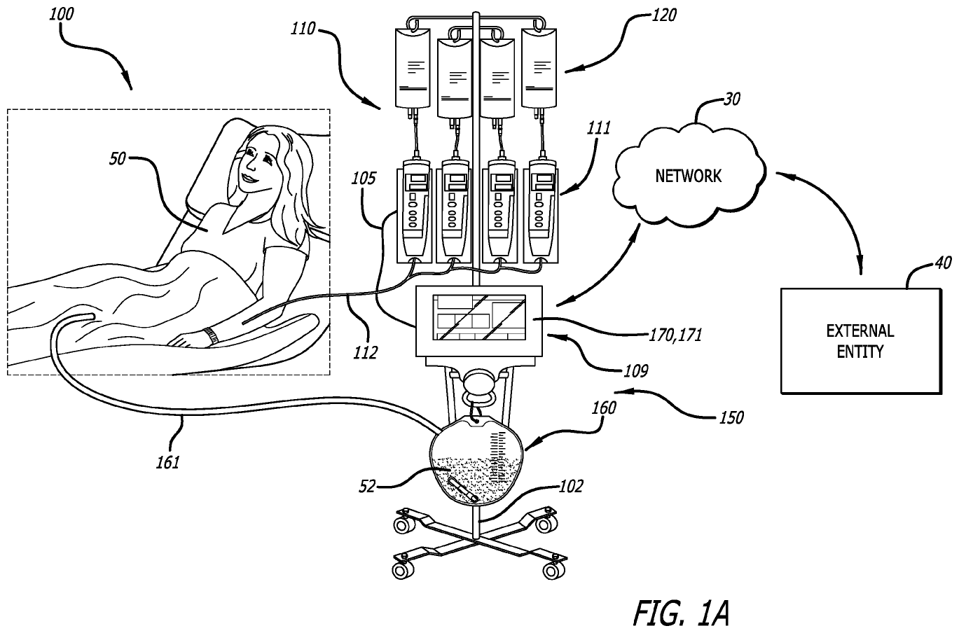

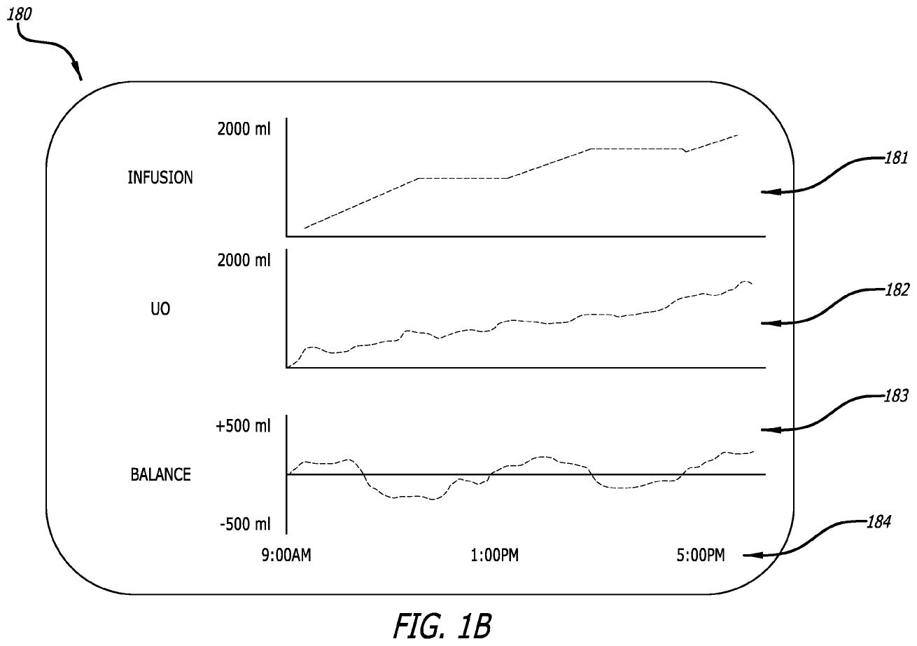

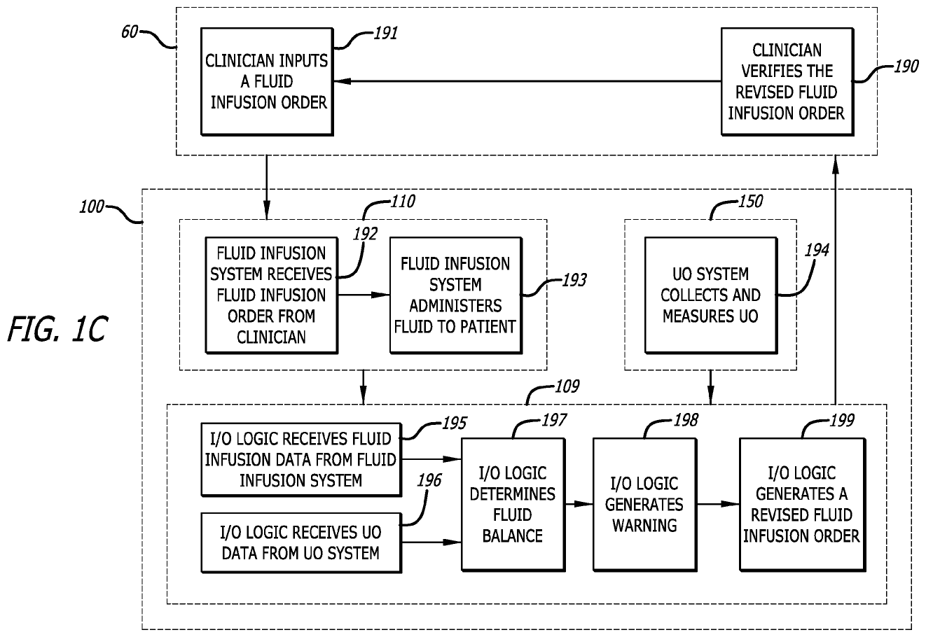

FIG. 1A illustrates an embodiment of a system for monitoring the input of fluids delivered to a patient along with monitoring the output of fluids from the patient, in accordance with some embodiments disclosed herein. The fluid I/O system 100 generally includes a fluid infusion system 110 and a urine output collection and measurement (UO) system 150. The fluid I/O system 100 is generally configured to automatically record (i) fluid infused into the patient 50 and (ii) fluid expelled from the patient 50 over a defined time period to define fluid input/output (I/O) data/information. The fluid I/O system 100 may then render I/O information on a display.

The fluid I/O system 100 is further configured to transmit the I/O data across a network 30 to an external entity 40. The network 30 represents the communication pathways between the fluid I/O system 100 and the external entity 40. In one embodiment, the network 30 is the Internet. The network 30 can also utilize dedicated or private communication links (e.g., WAN, MAN, or LAN) that are not necessarily part of the Internet. The network 30 may use standard communications technologies and/or protocols. The external entity 40 may be a person, an institution, or a cloud computing environment (e.g., cloud computing resources accessible via a network such as the internet). In some embodiments, the external entity 40 may include an electronic medical record (EMR).

In the illustrated embodiment, the fluid infusion system 110 includes one or more infusion pumps 111 configured to deliver medical fluids 120 to the patient 50 via one or more infusion lines 112. The fluid infusion system 110 is programmable to facilitate the infusion of the medical fluids 120 according to an infusion order. The infusion order may define the parameters of the fluid infusion, such as a medication, a fluid infusion volume, a rate of fluid infusion/delivery, and/or a start time of the infusion, for example. In some instances, the infusion order may include the infusion of multiple infusion fluids 120 (including multiple medications) over an extended period of the time, e.g., over a 24-hour period or longer, such as over multiple days.

In the illustrated embodiment, the UO system 150 includes a container 160 for collecting UO 52 (i.e., volume of urine) expelled from the patient 50 through a drainage tube 161 coupled with a catheter (not shown) inserted within the patient 50. The container 160 may be sized to contain a volume of urine 52 consistent with a total volume of infusion fluid 120 to be infused into the patient 50 according to the infusion order. The UO system 150 is configured to measure and record the volume of urine 52 collected within the container 160. A display module 170 of the UO system measures a weight of the UO 52 within container 160 over a defined time period, such as over a 24-hour period or longer. The display module 170 includes the hardware and software, including one or more processors and a non-transitory computer-readable storage medium, consistent with the measurement and recoding of the UO 52. The display module 170 includes a display 171 and in use, UO data/information may be rendered on the display 171.

The fluid I/O system 100 includes a communication link 105 between the fluid infusion system 110 and the UO system 150. In the illustrated embodiment, the communication link is a hard wire connection between the fluid infusion system 110 and the UO system 150. In other embodiments, the communication link 105 may be a wireless connection.

In the illustrated embodiment, the fluid infusion system 110 and the UO system 150 are each anchored to a support structure 102 (e.g., an IV pole) so that the fluid infusion system 110 and the UO system 150 may be handled/transported as a single unit. Having the fluid infusion system 110 and the UO system 150 attached to a single support structure 102 also occupies less space in the hospital room than each of the fluid infusion system 110 and the UO system 150 having separate support structures.

The fluid I/O system 100 includes fluid input/output (I/O) logic 109. The I/O logic 109 is configured to receive fluid infusion data from the fluid infusion system 110 and receive UO data from the UO system 150. The I/O logic 109 is further configured to generate I/O data/information for the patient 50 from the fluid infusion data and the UO data. The I/O information may include statistical values, tables, charts, graphs, and the like pertaining to the I/O data. The I/O logic 109 is configured to render the I/O data/information on the display 171.

The I/O logic 109 is also configured to transmit the I/O data/information across the network 30 to the external entity 40. As such, the clinician or other healthcare professional may access I/O data/information of the fluid I/O system 100 and thereby remotely monitor the fluid balance of the patient 50.

In some embodiments, the I/O logic 109 may be configured to generate an alert (e.g., a warning) in the event of an extreme I/O condition or trend. More specifically, the I/O logic 109 may compare the I/O data and/or statistical parameters with one or more limits stored in memory. As a result of the comparison, the I/O logic 109 may render one or more alerts on the display 171 and/or transmit the alert to the external entity 40. In one exemplary embodiment, one of the statistical parameters may be a fluid balance, i.e., a difference between an accumulated infusion volume of infusion fluid 120 and an accumulated volume of UO 52 over a defined time period. A positive fluid balance may indicate that the patient's renal function is unable to keep up with the fluid infusion rate. In an embodiment, the I/O logic 109 may compare the fluid balance with a high fluid balance limit and generate an alert when the fluid balance exceeds the high fluid balance limit. In another embodiment, the I/O logic 109 may compare the fluid balance with a low fluid balance limit (i.e., a high negative value) and generate an alert when the fluid balance exceeds the low fluid balance limit.

In some embodiments, the I/O logic 109 may be in the form of a software application that is loaded on the UO system 150 (i.e., stored in a non-transitory computer-readable storage medium of the UO system 150) and executable by hardware processing circuitry included therein. In other embodiments, the I/O logic 109, or a portion thereof, need not be loaded on the UO system 150 but may instead execute within a cloud computing environment (which may also be represented by the reference numeral 30) such that fluid infusion data and UO data are communicated to the I/O logic 109 for processing. Thus, any I/O logic 109 represented as being part of the UO system 150 may include an application programming interface (API) that is configured to transmit and receive data communication messages to and from the I/O logic 109 operating in the cloud computing environment.

Those of skill in the art will appreciate that the fluid I/O system 100 may contain other architectural modules that are not described herein. In addition, conventional elements, such as firewalls, authentication systems, network management tools, load balancers, and so forth are not shown as they are not material to the invention.

FIG. 1B illustrates an exemplary screen shot 180 depicting IO data as may be rendered on the display 171. The screen shot 180 may display an exemplary infusion graph 181. The infusion graph 181 shows the accumulated infusion volume of infusion fluid 120 delivered by the fluid infusion system 110 over a defined time period 184. The screen shot 180 may also display an exemplary UO graph 182. The UO graph 182 shows accumulated UO 52 collected and measured by the UO system 150 over the defined time period 184. The screen shot 180 may also display an exemplary fluid balance graph 183. The balance graph 183 shows a difference between the accumulated infusion volume of infusion fluid 120 and the accumulated UO 52. Observation of the balance graph 183 may provide the clinician with a fluid balance assessment of the patient 50, e.g., fluid overload versus dehydration.

FIG. 1C illustrates an exemplary process of monitoring the fluid balance of a patient 50, in accordance with some embodiments. In use, the clinician 60 may define and input a fluid infusion order into the fluid infusion system 110 (reference number 191). The fluid infusion order may include one or more fluid infusion parameters, such as a total volume of fluid to be infused, a rate of fluid infusion, and/or a medication dose, for example. The fluid infusion order is received by the fluid infusion system 110 (reference number 192) and the fluid infusion system 110 administers the infusion fluid to the patient 50 according to the order (reference number 193). During infusion, the UO system 150 collects and measures the UO 52 of the patient 50 (reference number 194). The I/O logic 109 of the fluid I/O system 100 receives (i) infusion data from the infusion system 110 (reference number 195), which infusion data may include the parameters of the fluid infusion order, and (ii) UO data from the UO system 150 (reference number 196).

The I/O logic 109 then determines (i.e., calculates) the fluid balance for the patient 50 (reference number 197). Calculating the fluid balance may include subtracting an accumulated volume of UO 52, as measured by the UO system 150, from an accumulated infusion fluid volume of infusion fluid 120. A positive fluid balance may indicate that the UO 52 is lagging behind (i.e., less than) the fluid infusion indicating that the patient 50 is retaining body fluid which in extreme cases may indicate fluid overload. A negative fluid balance may indicate that the UO 52 is over taking (i.e., greater than) the fluid infusion indicating that the patient 50 is failing to maintain a normal/desired amount of body fluid which in extreme cases may indicate dehydration of the patient 50.

As discussed above, the I/O logic 109 may define one or more limits related to the fluid balance of the patient 50. In the illustrated process, the I/O logic 109 may include high and low fluid balance limits, and the I/O logic 109 may compare the determined fluid balance with the fluid balance limits. In response to the comparison, the I/O logic 109 may generate an alert (reference number 198) when the fluid balance exceeds either limit. Generating the alert (reference number 198) may also include notifying the clinician directly via the display 171 and/or transmitting the alert to the external entity 40. In some embodiments, generating the alert includes informing the clinician if nephrotoxic drugs are being administered. For example, if there is a fluid imbalance, such as low urine output, the type of drug being administered can potentially be changed.

The I/O logic 109 may also generate a revised fluid infusion order (reference number 199) in accordance with the determined fluid balance. By way of example, in an instance of a positive fluid balance exceeding the high limit, the I/O logic 109 may generate a revised fluid infusion order that reduces the rate of fluid infusion, thereby causing the fluid balance to return to a desired value within a defined time period. In an alternative instance of a negative fluid balance exceeding the low limit, the I/O logic 109 may generate a revised fluid infusion order that increases the rate of fluid infusion, thereby causing the fluid balance to return to the desired value within a defined time period. It is to be noted, that the desired value for fluid balance may deviate from a zero value. In other words, the desired value for fluid balance may be positive or negative. In some instances, the revised fluid infusion order may include instituting an infusion, or alternating a rate of infusion, of a dose of medication (e.g., sodium) to chemically cause a change in the fluid balance of the patient 50.

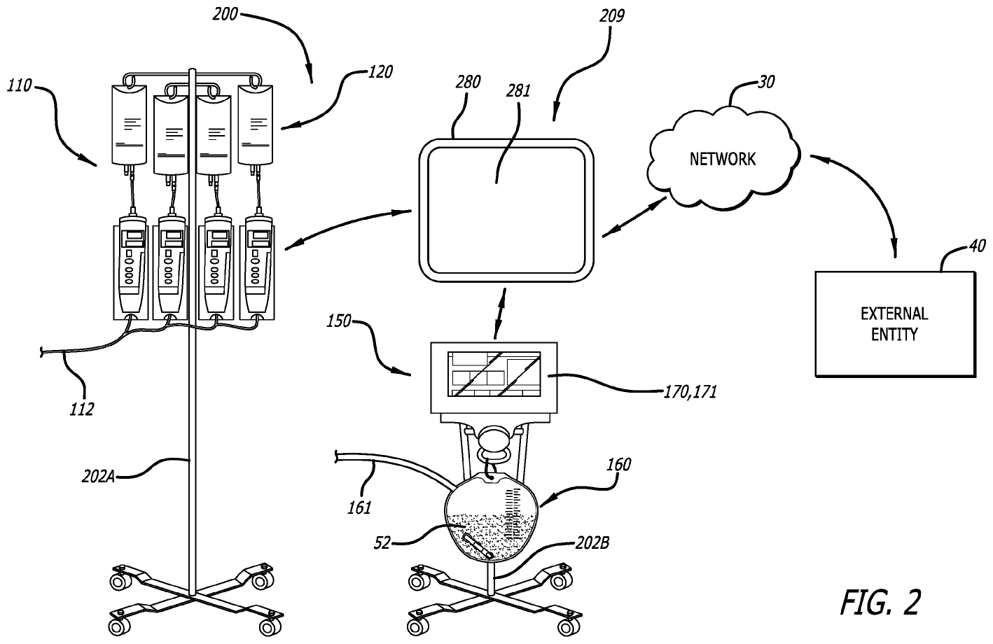

FIG. 2 illustrates a second embodiment of a body fluid (I/O) monitoring system 200, that can, in certain respects, resemble components of the fluid I/O system 100 described in connection with FIGS. 1A-1C. It will be appreciated that all the illustrated embodiments may have analogous features. Relevant disclosure set forth above regarding similar features thus may not be repeated hereafter. Moreover, specific features of the fluid I/O system 200 and related components shown in FIG. 2 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the system of FIG. 2. Any suitable combination of the features, and variations of the same, described with respect to the fluid I/O system 100 and components illustrated in FIGS. 1A-1C can be employed with the system and components of FIG. 2, and vice versa. This pattern of disclosure applies equally to further embodiments depicted in subsequent figures and described hereafter.

The fluid I/O system 200 includes the fluid infusion system 110 and the UO system 150. The UO system 150 is configured to measure and record the UO 52 collected within the container 160 and the display module 170 of the UO system 150 renders UO data on the display 171. The fluid I/O system 200 includes a separate display module 270 including a display 271. In some embodiments, the display module 270 may be a computing device, such as tablet computer, for example. The display module 270 is wirelessly coupled with the fluid infusion system 110 and the UO system 150.

The fluid I/O system 200 includes fluid input/output (I/O) logic 209 configured to (i) receive fluid infusion data from the fluid infusion system 110 and (ii) receive UO data from the UO system 150, (iii) render the I/O data on the display 271, and (iv) transmit the I/O data across the network 30 to the external entity 40. In some embodiments, the I/O logic 209 may be in the form of a software application that is loaded on the display module 270 and executable by hardware processing circuitry included therein. In other embodiments, the I/O logic 209, or a portion thereof, need not be loaded on the display module 270 but may instead execute within a cloud computing environment (which may also be represented by the reference numeral 30) such that fluid infusion data and UO data are communicated to the I/O logic 209 for processing. Thus, any I/O logic 209 represented as being part of the display module 270 may include an application programming interface (API) that is configured to transmit and receive data communication messages to and from the I/O logic 209 operating in the cloud computing environment.

As illustrated, in some embodiments, the fluid infusion system 110 may include a support structure 202A and the UO system 150 may include a separate support structure 202B. In other embodiments, the fluid infusion system 110 and the UO system 150 may be attached to single support structure, such as the support structure 202A, for example.

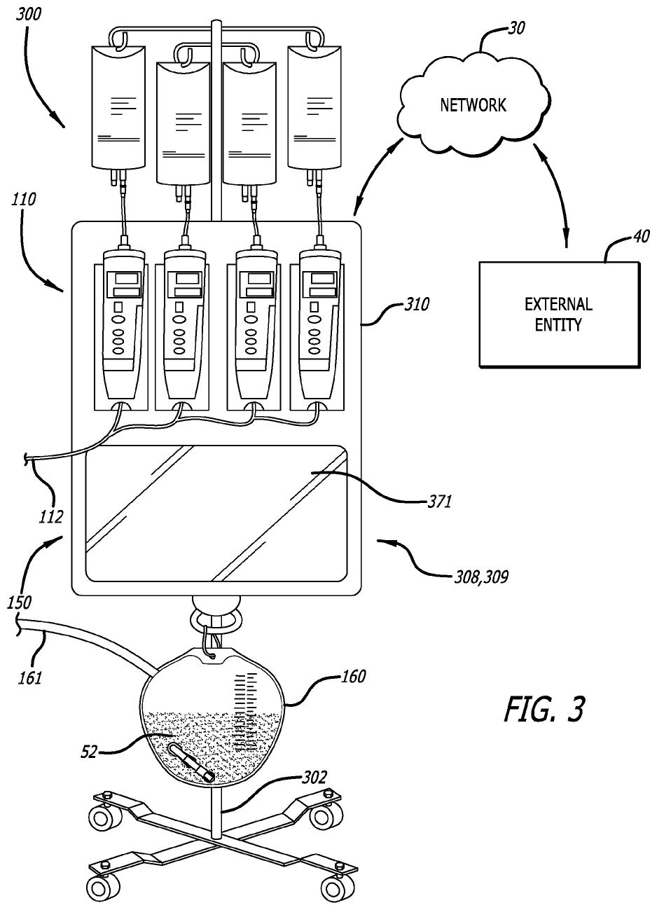

FIG. 3 illustrates a third embodiment of a body fluid (I/O) monitoring system 300. The fluid I/O system 300 includes the fluid infusion system 110 and UO system 150 disposed within a common housing 310 attached to a support structure 302. The fluid I/O system 300 further includes a display 371 incorporated into the housing 310, and the display 371 is configured to render I/O data/information thereon.

The fluid I/O system 300 includes fluid input/output (I/O) logic 309 configured to (i) receive fluid infusion data from the fluid infusion system 110 and receive UO data from the UO system 150, (ii) render the I/O data on the display 371, and (iii) transmit the I/O data across the network 30 to the external entity 40. In some embodiments, the I/O logic 309 may be in the form of a software application that is stored in a non-transitory computer-readable storage medium 308 of the fluid I/O system 300 and executable by one or more processors of the fluid I/O system 300. In further embodiments, the I/O logic 309 may be loaded on the fluid infusion system 110 or the UO system 150 and executable by hardware processing circuitry included within the respective system. In other embodiments, the fluid I/O system 300 may include an optional display module 370 disposed within the housing 310, and the I/O logic 309 may be loaded the display module 370 and executable by hardware processing circuitry included therein.

In still further embodiments, the I/O logic 309, or a portion thereof, may execute within a cloud computing environment (which may also be represented by the reference numeral 30) such that fluid infusion data and UO data are communicated to the I/O logic 309 for processing. Thus, any I/O logic 309 represented as being part of fluid I/O system 300 may include an application programming interface (API) that is configured to transmit and receive data communication messages to and from the I/O logic 309 operating in the cloud computing environment.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A system for monitoring the fluid balance of a patient, comprising:
a fluid infusion system configured to deliver an infusion fluid to the patient;
a urine output (UO) system configured to collect and measure a UO expelled from the patient, the UO system communicatively coupled with the fluid infusion system; and
a non-transitory computer-readable storage medium (CRM) including fluid input/output (I/O) logic, that when executed by one or more processors of the system, performs operations that include:
receiving fluid infusion data from the fluid infusion system,
receiving UO data from the UO system,
determining I/O data from the fluid infusion data and the UO data, and
rendering I/O data on a display of the system.

2. The system of claim 1, wherein the operations further include transmitting I/O data across a network to an external entity.

3. The system of claim 2, wherein the external entity includes an electronic medical record.

4. The system of claim 1, wherein the fluid infusion system is coupled with the UO system via a wired connection.

5. The system of claim 1, wherein the fluid infusion system and the UO system are attached together.

6. The system of claim 1, wherein the fluid infusion system and the UO system are anchored to a common support structure.

7. The system of claim 1, wherein the UO system includes the display.

8. The system of claim 1, wherein the UO system includes the I/O logic.

9. The system of claim 1, further comprising a housing, wherein the fluid infusion system and the UO system are disposed within the housing.

10. The system of claim 1, wherein the operations further include:
calculating a fluid balance from the I/O data,
comparing the fluid balance with a fluid balance limit stored in the CRM, and
generating an alert when the fluid balance exceeds the fluid balance limit.

11. The system of claim 10, wherein the operations further include transmitting the alert to the external entity.

12. The system of claim 10, wherein:
the infusion data includes an infusion order,
the operations include generating a revised infusion order from the I/O data, and
the alert includes the revised infusion order.

13. The system of claim 12, wherein the revised infusion order includes at least one of an increased or decreased rate of infusion.

14. The system of claim 12, wherein the revised infusion order includes an altered medical dose of the infusion fluid to chemically cause a change in the fluid balance.

15. The system of claim 12, wherein the revised infusion order includes a different drug to be administered.

16. A method for monitoring a fluid balance of a patient, comprising:
receiving fluid infusion data from a fluid infusion system, the fluid infusion system delivering infusion fluid to the patient;
receiving urine output (UO) data from a UO system, the UO system collecting and measuring UO from the patient;
determining fluid input/output (I/O) data from the fluid infusion data and the UO data; and
rendering the I/O data on a display, wherein:
the UO system is coupled with the fluid infusion system to define an I/O system, and
the display is coupled with the IO system.

17. The method of claim 16, further comprising transmitting the I/O data across a network to an external entity.

18. The method of claim 17, wherein the external entity includes an electronic medical record.

19. The method of claim 16, further comprising:
calculating a fluid balance from the I/O data;
comparing the fluid balance with a fluid balance limit stored in a non-transitory computer-readable storage medium of the I/O system; and
generating an alert when the fluid balance exceeds the fluid balance limit.

20. The method of claim 19, further comprising transmitting the alert to the external entity.

21. The method of claim 16, wherein the infusion data includes an infusion order, the method further comprising:
generating a revised infusion order from the I/O data; and
providing notification to a user of the revised infusion order.

22. The method of claim 21, wherein the revised infusion order includes at least one of an increased or decreased rate of infusion.

23. The method of claim 21, wherein the revised infusion order includes an altered medical dose of the infusion fluid to chemically cause a change in the fluid balance.

24. The method of claim 21, wherein the revised infusion order includes a different drug to be administered.
